(19) United States
(12) Patent Application Publication
(10) Pub. No.: US 2023/0301321 A1
(43) Pub. Date: Sep. 28, 2023

(54) SUPPLEMENTAL CACAO FERMENTATION

(71) Applicant: MycoTechnology, Inc., Aurora, CO (US)

(72) Inventors: James Patrick LANGAN, Aurora, CO (US); Marina NADAL, Aurora, CO (US); Anthony J. CLARK, Aurora, CO (US)

(73) Assignee: MycoTechnology, Inc., Aurora, CO (US)

(21) Appl. No.: 18/041,048

(22) PCT Filed: Aug. 25, 2021
(86) PCT No.: PCT/US2021/047560

§ 371 Date: Feb. 8, 2023

Related U.S. Application Data
(60) Provisional application No. 63/070,229, filed on Aug. 25, 2020.
(60) Provisional application No. 63/113,113, filed on Nov. 12, 2020.

(51) Int. Cl. A23G 1/02; A23G 1/48; C12N 1/14

(52) U.S. Cl. CPC A23G 1/02; A23G 1/48; C12N 1/14

(57) ABSTRACT

Provided is a method for production of a myceliated cacao product having reduced acidity, astringency, and/or bitterness tastes which optionally includes the steps of providing harvested cacao comprising beans, pulp and/or pods from the fruit pods of the species Theobroma cacao and fermenting the harvested cacao, preferably by a conventional fermentation process. In another step, a fungal inoculum comprising a filamentous fungus comprising, in one embodiment, Hericium erinaceus is used to inoculate the harvested cacao and the combination is cultured in a supplemental fermentation step for, for example, 1 to 2 days to produce the myceliated cacao product. The myceliated cacao product is optionally dried. In one aspect, a chocolate produced from the myceliated cacao product has reduced acidity, astringency, and/or bitterness tastes compared to a chocolate prepared from an unmyceliated cacao product. Products prepared by the methods described herein are also provided.

### CROSS-REFERENCE TO RELATED APPLICATIONS

This patent application claims the benefit of and priority to U.S. Provisional Patent Application No. 63/070,229 filed Aug. 25, 2020 and U.S. Provisional Patent Application No. 63/113,113, filed Nov. 12, 2020, each of which is incorporated hereby by reference in its entirety.

### BACKGROUND

Most of the world's cacao is grown in a band spanning 20 degrees of latitude north and south of the equator, as the cacao tree (Theobroma cacao) needs a humid, hot tropical environment in which to grow and fruit healthy cacao pods that contain the cacao bean. Harvested when ripe, the average cacao pod produces 20 to 50 cacao beans embedded in a mucilaginous pulp. Raw cocoa beans have an astringent, unpleasant taste and flavor, and have to be fermented, dried, and roasted to obtain the desired characteristic cocoa flavor and taste. The chocolate flavor is influenced by the origin of the cocoa beans, the cocoa cultivar, the on-the-farm fermentation and drying process, and the roasting and further processing performed by the chocolate manufacturers.

After removal of the beans from the pods, and/or splitting open the pods, the first step in cocoa processing is a spontaneous 3 to 10-day fermentation of beans and pulp (and/or pods) in heaps, boxes, baskets, vats, or trays. Typically, a microbial succession of yeasts, lactic acid bacteria, and acetic acid bacteria takes place during fermentation. The yeasts can help depectinise the pulp and produce ethanol from sugars and citric acid under anaerobic conditions in an acid, carbohydrate-rich environment. The ethanol, temperature and pH increase, causing bacteria to grow and convert sugars and organic acids into lactic acid. With increased oxygen, the ethanol initially produced by the yeasts is converted to acetic acid. Ethanol and acetic acid combined with heat cause mortality of the seed embryo and combine to develop the characteristic aroma, flavor, and color of the beans. These properties are further developed during drying, roasting, and final processing of well-fermented cocoa beans. However, despite this processing, bitter, astringent, and/or acid flavors can remain in the resultant chocolate, requiring use of added sugar and other components to reduce the bitterness and provide a sweeter flavor to the processed chocolate.

What is desired is a way of manufacturing cacao that achieves a great tasting product without the need for excessive sweetening. Further, it is desirable to rely on natural processes to achieve a great tasting product. Cacao is a complex food reported to contain over 20,000 different chemical compounds, making this a complex and hard-to-predict task.

### SUMMARY

In one embodiment, the present invention includes a method for production of a myceliated cacao product, which includes the step of providing a harvested cacao comprising beans, pulp and/or pods from the fruit pods of the species Theobroma cacao; and fermenting the harvested cacao. The method also includes providing a fungal inoculum comprising a filamentous fungus. In embodiments, the filamentous fungus can include a fungus selected from the group consisting of pure cultures of one or more of Inonotus obliquus, Hericium erinaceus, Cordyceps sinensis, Ganoderma lucidum, Pleurotus ostreatus, Pleurotus eryngii, Pleurotus citrinopileatus, Pleurotus djamor, Trametes versicolor, Lentinula edodes, Armillariella mellea, Tricholoma matsutake, Flammulina velutipes, Volvariella volvacea, Agaricus campestris, Agaricus blazei, Grifola frondosa, Pholiota nameko, Agrocybe cylindracea, Boletus edulis, Ganoderma applanatum, Hypsizygus marmoreus, Morchella hortensis, Morchella angusticeps, Morchella esculenta, Phellinus linteus, Auricularia auricula, Tremella fuciformis, Fomes fomentarius, Laetiporus sulfureus, Cordyceps sinensis, Cordyceps militaris, Cantharellus cibarius, and Polyporus umbellatus. In embodiments, the fungal inoculum comprises a pure culture of H. erinaceus. The harvested fermented cacao can then be inoculated with the fungal inoculum and cultured in a supplemental fermentation to allow the fungal inoculum to produce the myceliated cacao product. In embodiments, the supplemental fermentation takes place for between 6 hours and 2 days. The method may optionally include a drying step.

In embodiments, the myceliated cacao produced by the methods of the present invention produce a chocolate or chocolate liquor that has reduced acidity, astringency, and/or bitterness flavors, as compared to a chocolate prepared from an unmyceliated cacao product. Alternatively, such a chocolate or chocolate liquor has increased fruity flavor, increased nutty flavor, increased sweet caramel/malt flavor, compared with a chocolate produced from an unmyceliated cacao product.

The amount of fungal inoculum to inoculate the beans is optionally 10³ to 10⁴ colony forming units (CFU) per kg harvested cacao.

In embodiments, the myceliated cacao product is processed into chocolate or chocolate liquor.

In one embodiment, provided is a method for production of a myceliated cacao product, comprising the following steps: providing harvested cacao comprising beans, pulp and/or pods from the fruit pods of the species Theobroma cacao; fermenting the harvested cacao; providing a fungal inoculum comprising a filamentous fungus comprising Hericium erinaceus; inoculating the harvested cacao with the fungal inoculum at a level of about 10³ to 10⁴ colony forming units (CFU) per kg harvested and fermented cacao; culturing the harvested cacao and fungal inoculum in a supplemental fermentation step to allow myceliation to produce the myceliated cacao product for a time period of for 1 to 2 days; and drying the myceliated cacao product. In embodiments, a chocolate produced from the myceliated cacao product has reduced acidity, astringency, and/or bitterness tastes compared to a chocolate prepared from an unmyceliated cacao product.

The present invention includes myceliated products prepared by any of the methods disclosed herein, as well as a myceliated cacao product comprising cacao beans which has been cultured with a fungal culture comprising one or more of Inonotus obliquus or Hericium erinaceus, wherein a chocolate produced from the myceliated cacao product has reduced bitter tastes and/or reduced astringent tastes compared to an unmyceliated cacao product.

### DETAILED DESCRIPTION OF THE INVENTION

In an aspect, the present invention provides a method for the preparation of a myceliated cacao product. This method includes the step of providing harvested cacao, which can comprise one or more of the beans, pulp, and/or pods from the fruit pods of the species Theobroma cacao. The species Theobroma cacao as used herein comprises all varieties, particularly all commercially useful varieties, including but not limited to Criollo, Forastero, Trinitario, Arriba, and crosses and hybrids thereof. Cacao beans derived from the fruit pods of Theobroma cacao are the principal raw material for chocolate production. The cocoa beans are embedded in a mucilaginous pulp inside the pods. After the pods are harvested, the cocoa beans (usually including at least a portion of the surrounding pulp) are recovered from the pods. Accordingly, the plant material used in the method of the invention may preferably comprise cacao beans derived from the fruit pods of Theobroma cacao, and may further comprise the pulp derived from the said fruit pods. In an embodiment, the plant material may consist essentially of cocoa beans and the pulp derived from the fruit pods of Theobroma cacao. It is noted that the terms “cocoa” and “cacao” as used herein are considered as synonyms.

in an embodiment, the present invention relates to a supplemental fermentation that takes place during, before, after, or between, one or more of the fermentation and/or steps taking place in harvested cacao. In conventional chocolate production, after removal of the beans (and the surrounding pulp) from the pods, the beans and pulp are typically subjected to a spontaneous fermentation, which is important for the characteristics of the resulting cocoa, such as its aroma, flavor and color. Following the fermentation step is a drying step. It is thus an object of the present invention to provide a method for supplementing the naturally occurring (e.g., conventional) fermentation and/or drying of the harvested cacao to provide additional flavor benefits.

Fermentation is crucial to chocolate making, as it is the point at which beans develop the flavor precursors typically associated with cacao. Processes for a conventional cacao fermentation are well known in the art. Conventionally, fermentation of the cacao beans takes place when cacao pods are opened from harvested cacao and the cacao beans are removed (along with their mucilage), and placed in a heap, box, or basket, for example. The placenta inside the cacao pod may be removed. In one embodiment, the cacao beans and their mucilage may be poured onto banana leaves to form a heap of, for example, approximately 100 kg; or in other conventional processes, the pulp-coated seeds are placed into a fermentation box, traditionally lined with banana leaves. Fermentation typically takes place for 3-7 days depending on cacao type. The seeds are often called cacao beans at this stage, and some farmers sort cacao beans by size or pod color before fermenting. During fermentation, the sugars in the pulp surrounding the seeds are consumed by local yeast and bacteria, changing the composition of the beans. Temperatures inside fermentation boxes can reach 50 degrees C.; too high a temperature can damage the beans, but if the temperature is too low fermentation may not complete. Beans are usually turned multiple times during fermentation, to ensure that all cacao is equally fermented, and the temperature does not rise too high too quickly. Typically yeasts convert the sugars in the beans to ethanol, while lactic acid and acetic acid bacteria produce lactic acids and acetic acids which break down the beans. The heat that is produced also helps break down the cacao beans. The beans are typically occasionally stirred and monitored for temperature. Fermentation must take place long enough to reduce the astringency and bitterness and allow the acids to leave the beans, but over-fermentation may result in moldy flavors. Cut tests may be used to check the beans by slicing them open and checking the state of the beans; under-fermented beans have a typically purple appearance and over fermented beans may appear to be a very dark brown. Proper fermentation results in a darker brown (but not very dark) bean. Techniques to assess fermentation progress and completion are well-known in the art.

In some embodiments of the invention, this “natural” or self-starting e.g., conventional fermentation of cacao harvest is augmented by addition of starter cultures, optionally comprising yeasts and/or acid-producing bacteria such as lactic acid bacteria. Fermentations that are augmented in this fashion are also contemplated as part of the present invention. In some embodiments of the invention, a conventional fermentation step is skipped and the drying step takes place without a conventional fermentation step.

Drying the fermented beans is a conventional next step in the processing of the cacao harvest. Typically, after the conventional fermentation is deemed to be complete, the beans are poured onto tarps to be dried. The beans are conventionally spread in very thin layers, ideally in the sun, to discourage further fermentation with yeasts and bacteria from the fermentation step. Cacao conventionally dries in the sun for several days, and may be pulled under shelter during rainstorms and other adverse weather. If cacao dries too quickly, then it may develop a sour flavor due to trapped acids which would have otherwise escaped during the drying process. But if cacao dries too slowly then it may become moldy or begin to develop other off flavors. Once the moisture level of the beans is around 7%, the beans are put into bags and prepared for either storage or shipment.

As used herein, the term “fermentation” refers generally to any activity or process involving enzymatic decomposition (digestion) of organic materials by microorganisms. The term “fermentation” encompasses both anaerobic and aerobic processes, as well as processes involving a combination or succession of one or more anaerobic and/or aerobic stages. In the present invention, fermentation preferably involves the decomposition (digestion) of plant material. A “conventional” fermentation refers to typical or usual methods for fermenting harvested cacao beans to produce chocolate, for example. The term “supplementing” or “supplemental” fermentation as used herein in relation to the fermentation of harvested cacao encompasses but is not limited to use of one of the herein disclosed fungal species as a prepared fungal inoculum to initiate a fermentation process with that organism and/or initiating a particular stage of the fermentation process with that organism. In embodiments, the supplemental fermentation takes place before, during, or after a conventional fermentation. In some embodiments, the supplemental fermentation takes place after a conventional fermentation step. In embodiments, the supplemental fermentation step takes place before, after or during a drying step. In other embodiments, the supplemental fermentation takes place subsequent to a conventional fermentation step, and prior to a drying step.

By supplementing a conventional fermentation, the present invention allows for controlling or manipulating, by means of example and not limitation, the quality and/or quantity of both desirable and undesirable substances present in the myceliated cacao product, and taste and/or aroma characteristics of the myceliated cacao product and/or products obtained by further processing of the myceliated cacao product. In one embodiment, the taste of the myceliated cacao product made by processes disclosed herein is less acidic, less bitter and/or less astringent than a control conventional fermentation process that takes place in the absence of the supplemental fermentation. In other embodiments, a chocolate made from the myceliated cacao product has increased fruity flavor, increased nutty flavor, and/or increased sweet caramel/malt flavor, compared with a chocolate produced from an unmyceliated cacao product.

The methods of the invention also include the step of providing a fungal inoculum. The fungal inoculum to use with the present invention can be a filamentous fungi from phylum Basidiomycota and selected filamentous fungi from Ascomycota, including any fungi belonging to Polyporaceae and Hericiaceae, including the strains: Inonotus obliquus, Cordyceps sinensis, Hericium erinaceus, Pleurotus ostreatus, Pleurotus eryngii, Pleurotus citrinopileatus, Pleurotus djamor, Trametes versicolor, Lentinula edodes, Armillariella mellea, Tricholoma matsutake, Flammulina velutipes, Volvariella volvacea, Agaricus campestris, Agaricus blazei, Grifola frondosa, Pholiota nameko, Agrocybe cylindracea, Boletus edulis, Ganoderma lucidum, Ganoderma applanatum, Hypsizygus marmoreus, Morchella hortensis, Morchella angusticeps, Morchella esculenta, Phellinus linteus, Auricularia auricula, Tremella fuciformis, Fomes fomentarius, Laetiporus sulfureus, Bridgeoporus nobillismus, Cordyceps militaris, Cantharellus cibarius and/or, Polyporus umbellatus, and/or combinations thereof.

In embodiments, the fungal inoculum is a strain of Inonotus obliquus or Hericium erinaceus; a strain of Inonotus obliquus; or a strain of Hericium erinaceus.

Fungal inoculums useful in the present invention may be prepared by methods as known in the art. For example, in one embodiment, a pure strain of fungus is used. Fungal inoculums may be sourced commercially, in one embodiment, fungal cultures used are those commercially available from Pennsylvania State University (The Pennsylvania State University Mushroom Culture Collection, available from the College of Agriculture Sciences, Department of Plant Pathology and Environmental Microbiology, 117 Buckhout Laboratory, The Pennsylvania State University, University Park, Pa., USA 16802), and from Fungi Perfecti, PO Box 7634, Olympia, Wash. 98507, USA.

### Preparation of Fungal Inoculum for Inoculation

In one embodiment, methods for preparing the fungal inoculum to inoculate the cacao beans according to the invention include scaling up a fungal inoculum as defined herein in liquid culture. Such fungal inoculum which is readied for inoculation of the cacao beans in accordance with the invention is called a “fungal inoculum.”

In one embodiment, the fungal inoculum is in solid culture, such as an agar plate culture, grainspawn, or other known supportive substrate. In another embodiment, the prepared fungal inoculum is in liquid culture using soluble or partially-soluble media. A solid-culture inoculum may be resuspended in, for example, aqueous culture to form a slurry for ease of application. Liquid culture can be accomplished by any means known in the art and includes use of a bioreactor using conventional methods. The media to use in preparation of a fungal inoculum for use to inoculate the harvested cacao may be any suitable media known in the art. The inoculum may also be blended or powderized to break up any agglomerations or clumps.

### Inoculation Step

The method also comprises inoculating the harvested cacao with the fungal inoculum and culturing the harvested cacao and fungal inoculum together to effect myceliation to produce the myceliated cacao product before, after or during a conventional fermentation step or before, after or during a conventional drying step. These steps may be performed in any order. Optionally, the processes of the invention can include drying the myceliated cacao product (or, drying during a myceliation process) after the supplemental fermentation step by conventional drying steps, such as spreading beans and placing in the sun, for example, for five to fourteen days.

The fungal inoculum culture may be, e.g., a liquid culture, liquid pressed culture, frozen or dried form, including, e.g., freeze dried form and spray/fluid bed dried form, or frozen or freeze-dried concentrated. In one embodiment, the fungal inoculum culture may be a grainspawn culture, for example, on sorghum. In one embodiment, the grainspawn culture is blended to produce smaller particles prior to inoculation. The inoculum, if dried, may also be suspended in aqueous solution prior to inoculation. The culture may be packed in vacuum, or under an atmosphere of, e.g., N₂, CO₂ and the like. As a non-limiting example of inoculation according to the present invention, a bag of frozen grainspawn of a fungus described herein may be thawed in water. If spawn is not frozen, no thawing is necessary. If thawed, the spawn from the bag may be suspended in water as described herein. The suspension may be placed in a dispenser such as a watering can or sprayer attachment, then spread over or in the harvested cacao and mixed well with the harvested cacao by methods known in the art. Alternatively grainspawn may be a dried grainspawn that has been optionally blended and shaken out. The grainspawn may be mixed with water before application. Alternatively, a liquid culture (no grainspawn) may be used. The fermentations may then be covered as known in the art, for example, covered with banana leaves and then plastic foil to protect from rain, and fermented. Alternatively, the inoculation step may take place after the initial fermentation step and prior to, or during the drying step. The beans may be spread out and placed in the sun (or other typical methods for drying) and the inoculum may be spread on the beans during that step.

Inoculation of the prepared fungal inoculum into the harvested cacao may be carried out by any means known in the art, such as those known for inoculating harvested cacao heaps using starter cultures, see, e.g., US 2017/0311620, which is incorporated by reference in its entirety. In an embodiment, the cacao beans are agitated or stirred to help distribute the inoculum evenly and may be periodically stirred throughout the supplemental fermentation.

The fungal inoculum may optionally contain at least 10² colony forming units (CFU) per kg of cacao beans, e.g., fermented cacao beans. In embodiments, the inoculum may be applied at levels of between about 10² and 10⁶ CFU/kg cacao beans, or at least 10³ CFU/kg to about 10⁵ kg cacao beans, or, optionally at least about 10³ to about 10⁴ CFU/kg cacao beans.

In one embodiment, the present method for supplementing the fermentation of the harvested cacao may comprise adding to the harvested cacao the prepared fungal inoculum after a fermentation step, e.g., a conventional fermentation step.

Due to the increased temperatures of the beans during the conventional fermentation step, which may discourage growth of the fungal inoculum, in one embodiment, the inoculation takes place at a point after the conventional fermentation step, instead, taking place before and/or during the drying step. The inoculation can take place at any point after the fermentation step and before or during the drying step, but inoculation upon, or soon after, the fermentation step allows for myceliation of the beans to occur at lower temperatures which are suitable for the instantly disclosed fungal inoculum while moisture is still present.

In one embodiment the fermented beans are pasteurized prior to the fungal inoculum, but in preferred embodiments, the fermented beans are not pasteurized. The fermented beans after inoculation may be stored in a bag to provide a shaded environment and to delay drying until after the myceliation step is completed. In some embodiments, mycelial growth takes place during the supplemental fermentation step, e.g., the inoculation results in an increase in the amount of biomass present during the supplemental fermentation step. In other embodiments, mycelial growth does not occur after the inoculation step to any substantial amount, however the fungal inoculum is still metabolically active and results in the disclosed taste changes in the treated beans. The supplemental fermentation step may be carried out for anywhere between 1 hour and 10 days, but in one embodiment may be carried out for 24 to 48 hours. In embodiments, the supplemental fermentation may be carried out for about 1 hour, about 2 hours, about 4 hours, about 6 hours, about 8 hours, about 10 hours, about 12 hours, about 14 hours, about 16 hours, about 18 hours, about 20 hours, about 22 hours, about 24 hours, about 26 hours, about 28 hours, about 30 hours, about 32 hours, about 34 hours, about 36 hours, about 38 hours, about 40 hours, about 42 hours, about 44 hours, about 46 hours, about 48 hours, about 54 hours, about 60 hours, about 66 hours, about 72 hours, or more. Alternatively the supplemental fermentation may be carried out for about 10 hours to about 48 hours, about 18 hours to about 40 hours, about 24 hours to about 30 hours.

### Further Processing of Myceliated Product

In some embodiments, the myceliated product is optionally dried. Drying can be accomplished by means known in the art for drying cacao beans or other agricultural substrate. For example, myceliated product may be spread on a dry surface to dry in the sun using art-known methods. In one embodiment, the myceliated product is dried down to about an 8 to 9% moisture content.

Myceliated cacao can be subjected to conventional chocolate-processing steps to produce a range of cacao products, including chocolate. Accordingly the present invention can be used to make myceliated cacao mass, myceliated cacao butter, myceliated chocolate liquor, and all forms of chocolate derived thereof, as done by tempering, and other methods known in the art.

In one embodiment of the invention, myceliated cacao bean extract is used for nutraceuticals in an extract form, or in a powdered form achieved from dehydrating the extract. In another embodiment the myceliated cacao bean extract is used as an additive in functional foods to bolster flavor and nutrient content. This extract can be in aqueous form, infused in an alcohol base, or in vegetable glycerin, for example. A powdered form can be achieved from dehydrating the extract. The powdered form can be added to any packaged food. An example of a functional food includes a bottled drink, a snack bar, or a high-protein powder mixture used to make protein shakes. The powdered form can also be used in ice-cream.

Myceliated cacao in some embodiments can be used to produce chocolate or liquor that have flavors that are less acidic, less bitter, and/or less astringent than conventionally fermented cacao. In some embodiments, the myceliated cacao can be used to produce chocolate or liquor that have increased fruity flavor, increased nutty flavor, and/or increased sweet caramel/malt flavor. Accordingly, one benefit of using the myceliated cacao is that a low sugar (or sweetener) version of any food including cacao can be made to taste as good as compared with conventionally-fermented cacao having more sweeteners (i.e. sugar, stevia, or synthesized sweeteners).

In an embodiment, a chocolate or chocolate liquor made from the myceliated cacao produced by methods of the invention have reduced acidic flavors, and/or reduced bitterness and/or reduced astringent flavors, compared unmyceliated cacao. In an embodiment, the compositions of the invention have the changed organoleptic perception as disclosed in the present invention, as determined by human sensory testing. It is to be understood that the methods of the invention only optionally include a step of determining whether the flavors or aromas of the compositions of the invention differs from a control material. The key determinant is, if measured by methods as disclosed herein, that the compositions of the invention are capable of providing the named differences from control materials which have not been combined, mixed or treated as described in the present invention.

Sensory evaluation is a scientific discipline that analyses and measures human responses to the composition of food and drink, e.g. appearance, touch, odor, texture, temperature and taste. Measurements using people as the instruments are sometimes necessary. The food industry had the first need to develop this measurement tool as the sensory characteristics of flavor and texture were obvious attributes that cannot be measured easily by instruments. Selection of an appropriate method to determine the organoleptic qualities, e.g., flavor, of the instant invention can be determined by one of skill in the art, and includes, e.g., discrimination tests or difference tests, designed to measure the likelihood that two products are perceptibly different. Responses from the evaluators are tallied for correctness, and statistically analyzed to see if there are more correct than would be expected due to chance alone.

In the instant invention, it should be understood that there are any number of ways one of skill in the art could measure the sensory differences.

In an embodiment, the products produced from the myceliated cacao product(s), e.g., produced by methods of the invention, have reduced acidic flavors, reduced bitterness, reduced astringency, increased fruity flavor, increased nutty flavor, and/or increased sweet caramel/malt flavor. Such methods include change in taste threshold, change in intensity, and the like. At least 10% or more change (e.g., reduction in) is preferred. The increase in desirable flavors and/or tastes may be rated as an increase of 1 or more out of a scale of 5 (1 being no taste, 5 being a very strong taste.) Or, a reference may be defined as 5 on a 9 point scale, with reduced at least one flavor or taste as 1-4 and increased flavor or taste as 6-9. The invention includes reduction in one or more of the named organoleptic qualities (bitter tastes, grassy tastes, pea tastes and/or other undesirable flavors) as discussed herein.

Additionally, the organoleptic qualities of the compositions of the invention may also be improved by processes of the current invention. For example, deflavoring can be achieved, resulting in a milder flavor and/or with the reduction of, for example, bitter and/or astringency and/or other flavors. The decrease in undesirable flavors and/or tastes as disclosed herein may be rated as a decrease of 1 or more out of a scale of 5 (1 being no taste, 5 being a very strong taste.)

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. The term “consisting of” is construed as a close-ended term (i.e., excluding components or steps other than those listed). The term “consisting essentially of” allows for the inclusion of components or steps that are not essential to the function or activity of the product or method and do not materially affect the function or activity.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The following examples are provided for illustrative purposes only and are not intended to limit the scope of the invention.

### EXAMPLES

### Example 1

Specific and pure strains of fungi obtained from reference collections were manipulated in sterile environments in all Examples herein.

Separate 1 L Erlenmeyer flasks were filled with 300 mL of liquid containing glucose and yeast extract. The flask was sterilized in an autoclave and each inoculated with one of a stock culture (on petri plates) of C. sinensis, I. obliquus, H. erinaceus, and G. lucidum. The flask was set on an incubated shaker cabinet at 120 RPM and 16° C. for two weeks. Once confirmed that the flask was free of contamination, flasks were slightly blended with a sterile blender, and 10 mL was propagated into 800 g of prepared, sterile sorghum grain. This sorghum grain had 1 part of RO water added to it prior to sterilization, so the total mass of sorghum initially used was 400 g with 400 mL RO water added. This grain was incubated at 16° C. for one month to prepare the grain spawn.

Once colonized, the grain was blended with RO water at 1:10 w/v (grain:water), 1:100 w/v (grain:water), 1:1000 w/v (grain:water), or 1:10,000 w/v (grain:water).

### Example 2

Harvested cacao was placed in a heap, and sprayed with a 1:10,000 diluted grain spawn in a proportion of 1 L diluted grain spawn per kg harvested cacao and allowed to myceliate for five days at ambient temperatures. The myceliated cacao is then dried, and following drying, is roasted, winnowed, and the nibs are used to prepare into a chocolate product using conventional processes.

### Example 3

Five separate piles of 80 kg of harvested cacao, immediately after harvest, is placed in heaps. 4 L of each of the diluted grain spawn described in Example 1 are applied to the heaps by spraying, followed by mixing the heap. A control heap is sprayed with RO water. The heaps are allowed to myceliate under conditions that also allow a conventional fermentation for five days. When the conventional fermentation is complete, the beans are dried to an 8 to 9% moisture content using conventional processes, then are roasted and winnowed, using conventional processes. The nibs are obtained and tasted and in all samples, e.g., C. sinensis, I. obliquus, H. erinaceus, and G. lucidum-supplemented fermentations, the nibs are less bitter than the nibs yielded by the control process.

### Example 4

Five separate piles of 80 kg of harvested cacao, immediately after harvest, is placed in heaps. The heaps are allowed to ferment under conditions that also allow conventional fermentation for five days. When the conventional fermentation is complete, the beans are spread out to dry in the sun under conventional conditions, and 4 L of each of the diluted grain spawn described in Example 1 are separately applied to the spread beans by spraying, and allowed to air dry to an 8 to 9% moisture content over the course of several days. The beans are then are roasted and winnowed, using conventional processes. The nibs are obtained and tasted and in all samples, e.g., C. sinensis, I. obliquus, H. erinaceus, and G. lucidum-supplemented fermentations, the nibs are less bitter than the nibs yielded by the control process.

### Example 5

Different grainspawn cultures using either Inonotus obliquus (Chaga mushroom mycelia) and Hericium erinaceus (lion's mane mushroom mycelia) were prepared as follows. 1 lb of sorghum was blended with 450 mL of deionized distilled water, placed in a sterile bag, and sterilized in an autoclave using standard sterilization techniques (1 hour, 121° C. by autoclave). After cooling, each of the bags were inoculated with 10 mL a liquid inoculum of the mycelial mass at approximately 10⁵ cfu/ml. The bags were stored at room temperature for two to four weeks, with occasional agitation of the bags, for even myceliation. At the end of the myceliation the bags contained about 10⁴ cfu/g sorghum. When myceliation was complete, the colonized sorghum was removed from the bags and air-dried in a sterile location until the sorghum had 5-6% moisture content, about 2 to 3 days. The dried myceliated sorghum was then blended in a blender (sterile) and stored until use.

### Example 6

Cacao beans were harvested and fermented conventionally using art-known techniques for harvesting and fermenting cacao beans. After the fermentation step, 5.9 kg of wet beans (approximately 3 lbs dried), were divided into pasteurized lots (15 minutes at 165° F.) or non-pasteurized lots. Cacao beans then were inoculated with 3.8 g/kg of myceliated ground sorghum containing live Hericium erinaceus (lion's mane mushroom mycelia) as prepared in Example 5, or with the equivalent amount of Hericium erinaceus (lion's mane mushroom live mycelia) delivered in a liquid suspension in water. Control beans were inoculated with sterile inoculum. Inoculated beans were then placed into chlorox-wiped black woven polypropylene bags in shade to provide for darkness. Beans in both the non-pasteurized and the pasteurized lots were myceliated for either one day (24 hours), 2 days (48 hours), or three days (64 hours) in the bags. The cacao beans were turned once a day to assist in distribution of mycelia. At the end of the myceliation period, beans were placed on a rack in sun until <8% moisture was achieved, or between 2 to 7 days. Dried beans were then roasted and winnowed using standard procedures to produce the cacao nibs, then a chocolate liquor was prepared and processed into a dark chocolate using standard techniques from each of the test samples.

Sensory assessment of the dark chocolate was undertaken. The non-pasteurized sample (1 day myceliation) and the pasteurized sample (1 day myceliation) was preferred (in that order) over the control (non-myceliated) as these samples, particularly the non-pasteurized sample, had decreased acidity, decreased astringency, decreased bitterness flavors, as compared to the control. The chocolate prepared from these myceliated samples also had increased fruity flavor, increased nutty flavor, increased sweet caramel/malt flavor, and overall “global quality” compared the control. The 2 day and 3 day myceliation samples, either pasteurized or not pasteurized, were not preferred over the control.

1. A method for production of a myceliated cacao product, comprising:
a) providing harvested cacao comprising beans, pulp and/or pods from the fruit pods of the species Theobroma cacao;
b) conventionally fermenting the harvested cacao;
c) providing a fungal inoculum comprising a filamentous fungus;
d) inoculating the harvested cacao with the fungal inoculum; and
e) culturing the harvested cacao and fungal inoculum as a supplemental fermentation step to allow myceliation to produce the myceliated cacao product.

2. The method of claim 1, wherein the step of fermenting the harvested cacao takes place prior to the inoculation step.

3. The method of claim 2, wherein the method further includes a drying step that takes place subsequent to the inoculation step.

4. The method of claim 2, wherein the culturing step e) takes place for 1 to 2 days.

5. The method of claim 1, wherein a chocolate produced from the myceliated cacao product has reduced acidity, astringency, and/or bitterness tastes compared to a chocolate prepared from an unmyceliated cacao product.

6. The method of claim 1, wherein a chocolate produced from the myceliated cacao product has increased fruity flavor, increased nutty flavor, and/or increased sweet caramel/malt flavor, compared with a chocolate produced from an unmyceliated cacao product.

7. The method of claim 1, wherein the fungal inoculum is an inoculum comprising a fungus selected from the group consisting of pure cultures of one or more of Inonotus obliquus, Hericium erinaceus, Cordyceps sinensis, Ganoderma lucidum, Pleurotus ostreatus, Pleurotus eryngii, Pleurotus citrinopileatus, Pleurotus djamor, Trametes versicolor, Lentinula edodes, Armillariella mellea, Tricholoma matsutake, Flammulina velutipes, Volvariella volvacea, Agaricus campestris, Agaricus blazei, Grifola frondosa, Pholiota nameko, Agrocybe cylindracea, Boletus edulis, Ganoderma applanatum, Hypsizygus marmoreus, Morchella hortensis, Morchella angusticeps, Morchella esculenta, Phellinus linteus, Auricularia auricula, Tremella fuciformis, Fomes fomentarius, Laetiporus sulfureus, Cordyceps sinensis, Cordyceps militaris, Cantharellus cibarius, and Polyporus umbellatu.

8. The method of claim 5, wherein the fungal inoculum comprises a pure culture of H. erinaceus.

9. The method of claim 5, wherein the fungal inoculum comprises a pure culture of I. obliquus.

10. The method of claim 1, wherein the step of inoculating comprises inoculating with a grainspawn culture.

11. The method of claim 10, wherein the grainspawn culture is applied in an aqueous slurry.

12. The method of claim 11, wherein the amount of fungal inoculum is 10³ to 10⁴ colony forming units (CFU) per kg harvested cacao.

13. The method of claim 1, further comprising roasting the myceliated cacao product.

14. A method for production of a myceliated cacao product, comprising:
a) providing harvested cacao comprising beans, pulp and/or pods from the fruit pods of the species Theobroma cacao;
b) conventionally fermenting the harvested cacao;
c) providing a fungal inoculum comprising a filamentous fungus comprising Hericium erinaceus;
d) inoculating the harvested cacao with the fungal inoculum at a level of about 10³ to 10⁴ colony forming units (CFU) per kg harvested and fermented cacao;
e) culturing the harvested cacao and fungal inoculum to allow myceliation to produce the myceliated cacao product for a time period of for 1 to 2 days as a supplemental fermentation step; and
f) drying the myceliated cacao product;
wherein a chocolate produced from the myceliated cacao product has reduced acidity, astringency, and/or bitterness tastes compared to a chocolate prepared from an unmyceliated cacao product.

15. A myceliated product prepared by the method of claim 14.

16. A myceliated cacao product comprising cacao beans which has been cultured in a supplemental fermentation step with a fungal culture comprising one or more of Inonotus obliquus or Hericium erinaceus, wherein a chocolate produced from the myceliated cacao product has reduced bitter tastes and/or reduced astringent tastes compared to an unmyceliated cacao product.

17. The myceliated cacao product of claim 16, wherein the myceliated cacao product was inoculated with the fungal culture subsequent to a fermentation step and prior to a drying step.

18. The myceliated cacao product of claim 16, wherein the fungal culture is H. erinaceus.

19. The myceliated cacao product of claim 16, wherein the fungal culture was inoculated into the cacao beans is 10³ to 10⁴ CFU/kg fermented beans and allowed to culture for 1-2 days.

20. A myceliated product prepared by the method of claim 1.
